(19) United States
(12) Patent Application Publication
(10) Pub. No.: US 2026/0069730 A1
(43) Pub. Date: Mar. 12, 2026

(54) ADD-ON MODULE TO DOOR ACCESS DEVICES UTILIZING UVC TO DESTROY AIRBORNE PATHOGENS

(71) Applicant: Tom Chi, Wichita, KS (US)

(72) Inventor: Tom Chi, Wichita, KS (US)

(21) Appl. No.: 19/108,517

(22) PCT Filed: May 1, 2023
(86) PCT No.: PCT/US2023/020607

§ 371 Date: Mar. 4, 2025

(30) Foreign Application Priority Data
May 4, 2022 (US) 63/338316

(51) Int. Cl. A61L 2/10; A61L 2/24; A61L 9/20; E05B 15/00

(52) U.S. Cl. CPC A61L 2/10; A61L 2/24; A61L 9/20; E05B 15/00; A61L 2103/00; A61L 2202/14; A61L 2209/111

(57) ABSTRACT

An ultraviolet C self-sterilizing germicidal light airborne pathogen killing system for ultraviolet C self-sterilization of door access devices, such as door handles, door levers, door push plates for hand sanitation, which includes a door access device associated with a door jamb, a module affixed to the door access device, a source of electricity associated with the door access device, a device for electrically powering the module, a device for the module to sanitize air for hand sanitization, and a device for activating the module for hand sterilization. The system may be of varying sizes depending on room measurements/needs and the module may contain its own batteries, may run on direct electrical wiring using a concealed power transfer system or may use any related battery or power source. The module may also be functional when utilized as a stand-alone device without being associated with door access devices.

### BACKGROUND OF THE INVENTION

### Technical Field of the Invention

The present invention relates, generally, to door access devices that are both self-sterilizing and further equipped with an add-on module that sterilizes the surrounding air. These modules and their “host” UVC door levers devices will reduce mortality, as well as substantially reduce the funds spent on these diseases, making the world a safer place and more prosperous place.

It is well-established that germs from hands are easily communicated to a person's face, especially when the hands touch the face often several times per minute, as for example, when the eyes are rubbed, the nose is touched and other times when the hands consciously and unconsciously touch the face. Additionally hospital acquired infections (“HAI”) result in over 1 million deaths annually worldwide and over $200 billion is spent yearly to remediate the problem.

### Description of the Prior Art

The prior art known to the inventor pertaining to ultraviolet sanitizing technology includes Visco et al., U.S. Pat. No. 4,856,140, issued Aug. 15, 1989; Manson et al., U.S. Pat. No. 6,289,557, issued Sep. 18, 2001; Jones, U.S. Pat. No. 7,175,807, issued Feb. 13, 2007; Sassoon, U.S. Pat. No. 7,360,674, issued Apr. 22, 2008; U.S. Pat. No. 7,458,742, Stropkay et al., issued Dec. 2, 2008; Jones, U.S. Pat. No. 7,598,501, issued Oct. 6, 2009; Zevallos, U.S. Pat. No. 7,716,789, issued May 18, 2010; Balzano, U.S. Pat. No. 8,353,085, issued Jan. 15, 2013; Caron, U.S. Pat. No. 8,375,521, issued Feb. 19, 2013; Bracey, U.S. Pat. No. 9,051,757, issued Jun. 9, 2015; Roberts et al., U.S. Pat. No. 9,376,833, issued Jun. 28, 2016; and, Strombeck et al., U.S. Pat. No. 10,422,158, issued Sep. 24, 2019.

It is desirable to provide a system to eliminate airborne pathogens, which may be breathed in or otherwise transmitted to people and spreading sick and even death, which is not addressed by the prior art.

### SUMMARY OF THE INVENTION

An object of the present invention is to provide a system for sanitizing door access devices such as door handles to prevent transmission of pathogens from door handles to a person's hand and subsequently to a person's face.

A further object of the present invention is to provide a way to disinfect door handles for the prevention of pathogens to contact a person and spread disease and other unsanitary conditions.

An additional object of the present invention is to provide an ultraviolet C self-sterilizing germicidal light airborne pathogen killing module associated with a door handle for ultraviolet C self-sterilizing door handle/lever access devices and the like for hand sanitation.

Another object of the present invention is to utilize LED as a source of ultraviolet C self-sterilizing germicidal light for hand sanitization in the form of a module that is associated with a door handle/lever that kills airborne pathogens.

Broadly, the foregoing and related objects are achieved by the present invention, which pertains to self-sterilizing door access devices such as UVC equipped door handles and the like which are touched by a user's hand and made safe within a few seconds by the UVC light in the door lever, push/pull systems, elevator button etc. by adding onto the door access device a module containing a source of ultraviolet C self-sterilizing germicidal light.

More particularly, the present invention achieves the foregoing objects by broadly providing an ultraviolet C self-sterilizing germicidal light airborne pathogen killing system for ultraviolet C self-sterilization of door access devices such as door handles, door levers, door push plates for hand sanitation, which includes a door access device associated with a door jamb, a module affixed to the door access device, a source of electricity associated with the door access device, means for electrically powering the module, means for the module to sanitize air for hand sanitization, and means for activating the module for hand sterilization.

The add-on device (module) described herein also uses UVC to destroy airborne pathogens and said module combined with the UVC door levers and other devices which work for touch surface contamination and sterilization adds a component for sterilizing the air in areas proximal to the UVC door lever and instant add-on device. The module makes the surrounding air safer to breathe by destroying airborne pathogens which may (or may not) be touch-surface transmissible and this additional method of sterilization combined with the existing UVC door lever, etc. creates both a touch-safe and air-safe environment.

As used throughout this Specification, “UVC” is intended to signify (ultraviolet C light), UVC LEDs, CCT (cold cathode tubes), or other methods of light or electro-magnetic sterilization.

Scientifically, the term “sterilization” is a bit different from the actual mechanism of sanitization as, more specifically, the UVC damages pathogens'DNA causing the pathogens not to be able to reproduce, which in turn eliminates their ability to infect and make persons ill.

These add-on modules may utilize UVC LEDs or CCT (cold cathode tubes) or other methods of light or electromagnetic sterilization. Air enters the device from a port on the desired side, front or bottom of the module and then passes through channels or otherwise optimized pathway enabled with UVC light that kills pathogens and then the pathogen-free air exits the module and re-enters the surrounding room. The module may be sized according to treatment needs. For example, a small hospital bathroom may need a small air treatment module attached to the UVC door lever/knob/handle, etc. and the opposite side of the door may be located in a larger room, hence a larger module may be required and used.

The devices may share batteries or direct power that is also utilized by the UVC door lever or the modules may have its own batteries or direct power transfer from the door jamb using concealed power transfer or other method which provides power to the module.

The module may perform other tasks such as one-or two-way audio, video, etc., so that installing these devices increases ability to reach out to nearby people to advise of emergencies, for them to request entry and a multitude of other natural uses and the video/audio may be by hard-wiring as the power may potentially also use or Bluetooth, wi-fi or other means. When combined with other door levers/modules the total effect of an entire building's air (and touch-surface levers, etc.) become harmless for users.

The ultraviolet C germicidal light airborne pathogen killing module may be battery-powered or directly wired to an external power source using a concealed power transfer system and may be mounted above, below or piggybacked onto battery or directly to lever housing. The module contains pathways with baffles, dividers or tube/channels for air to enter, circulate within the module and exit after sterilization by UVC LED or cold cathode tube technology.

In a preferred embodiment of the present invention the module is energized by snapping onto an electrically powered UVC door lever that receives its power from an electrically energized member in the door jamb and in a second preferred embodiment the module is energized by batteries.

CCT/LEDs may be located inside the main housing of the module and/or may be located in module housing cover or imbedded in same and the module and may be sized according to room needs and positioned on either side of door.

The module may contain a camera with wireless or wired options for viewing by cell phone or computer containing a speaker for announcements and be controlled locally, manually or distally by remote programming method.

The module may contain motion or other sensors as desired so that it activates on detecting motion.

It is within the scope of the present invention to combine UVC door levers, battery solutions and concealed power transfer systems with UVC air sanitation units for creating a synergy of UVC pathogen-killing devices to eliminate presence of MRSA, Covid-19 and other pathogens, both in the air and on the most frequently touch surfaces such as door levers where 23% of MRSA is found in endemic situations in hospitals.

This modular device combines a door lever with (or without) UVC sanitation with a UVC air sanitation module, which may be operable by batteries or may operable on DC voltage via a concealed power transfer systems, which brings power from the door jamb side of the door, through the door set and to the door lever and/or UVC air sanitizer.

The invention may be programmed/controlled directly on the device or using a phone/computer app. Device may be sized according to spatial needs (larger/smaller) and multiple units on multiple doors can work together to sanitize larger spaces. The invention may also be combined with a charcoal or other filter for bathroom air sanitation (e.g., odors, etc).

An important concept of the present invention is the replacement of a conventional door set and combines modular options of UVC in the door lever/knob/push plate etc and the optional use of a UVC air filtration modular, which is also powered with the UVC door lever directly by concealed power transfer or by battery.

Other objects and features of the present invention will become apparent when considered in combination with the accompanying drawing figures, which illustrate certain preferred embodiment of the present invention. It should, however, be noted that the accompanying drawing figures are intended to illustrate only select preferred embodiments of the claimed invention and are not intended as a means for defining the limits and scope of the invention.

### BRIEF DESCRIPTION OF THE DRAWING FIGURES

In the drawing figures, wherein similar features are denoted with similar reference numerals throughout the several views:

FIG. 1 is a perspective view of the ultraviolet C self-sterilizing germicidal light airborne pathogen killing system, with assembled module A1, for ultraviolet C self-sterilization of door access devices such as door handles, door levers, door push plates for hand sanitation with the invention assembled to door 12;

FIG. 2 is a perspective view of the ultraviolet C self-sterilizing germicidal light airborne pathogen killing system, with assembled module A1, for ultraviolet C self-sterilization of door access devices such as door handles, door levers, door push plates for hand sanitation with the invention assembled to door 12 with the module displaced;

FIG. 3 is an elevation view of the ultraviolet C self-sterilizing germicidal light airborne pathogen killing system, with cut-away view assembled module A1, for ultraviolet C self-sterilization of door access devices such as door handles, door levers, door push plates for hand sanitation with the invention assembled to door 12; and,

FIG. 4 is a partial elevation view of the ultraviolet C self-sterilizing germicidal light airborne pathogen killing system, with a cut-away view of module A1 for ultraviolet C self-sterilization of door access devices such as door handles, door levers, door push plates for hand sanitation.

### DESCRIPTION OF THE REFERENCED NUMERALS

Turning now descriptively to the drawings, in which similar reference characters denote similar elements throughout the several views, the figures illustrate the use of the present invention. With regard to the reference numerals used, the following numbering is used throughout the various drawing figures:
A1 module of present invention attached to door access device
B power transfer contacts in both door 12 and power transfer assembly B1 in door jamb 12a
B1 power transfer assembly in door jamb to energize the UVC door access device
C module snap-on joiner electrical connection attaching means on top of module
C1 module snap-on joiner electrical connection attaching means on top of UVC door access device
D air entry ports for ingestion of untreated air
D1 untreated air
E air exit ports for UVC treated air
E1 UVC treated air
F fan
G baffles-pathways with dividers or tube/channels for un-sanitized air to enter, circulate within module and exit after sterilization by UVC LED or cold cathode tube technology
H sources of ultraviolet C self-sterilizing germicidal light
10a door handle
12 door
12a electrified door jamb in wall
14 motion sensor
14a optical sensor
16 heat sensor
18 battery for alternate power source
20 camera
22 speaker
24 remote programing system
26 exposed section to facilitate the functioning of internal components

### DETAILED DESCRIPTION OF THE DRAWING FIGURES AND PREFERRED EMBODIMENTS

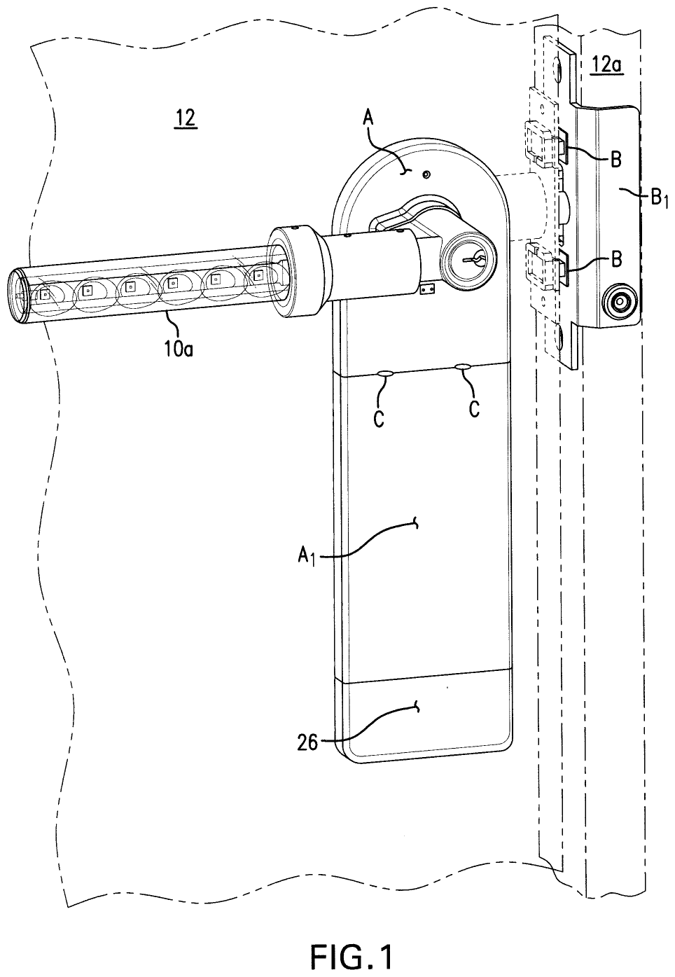

FIG. 1 is a perspective view of the ultraviolet C self-sterilizing germicidal light airborne pathogen killing system A, with assembled module A1, for ultraviolet C self-sterilization of door access devices, such as door handles, door levers, door push plates and alike for hand sanitation with the present invention assembled to door 12. A handle 10a may be as illustrated and/or in the shape of a lever.

UVC door lever system A is affixed to door 12 and communicates and receives electrical power from power transfer assembly B1 in door jamb 12a by power transfer contacts B in both door 12 and power transfer assembly B1 in door jamb 12a. Module A1 is attached to UVC door lever system A by module snap-on joiner electrical connection means C on top of module A1. Module A1 contains the elements of instant invention 10 with exposed section 26 to facilitate the functions of its internal components.

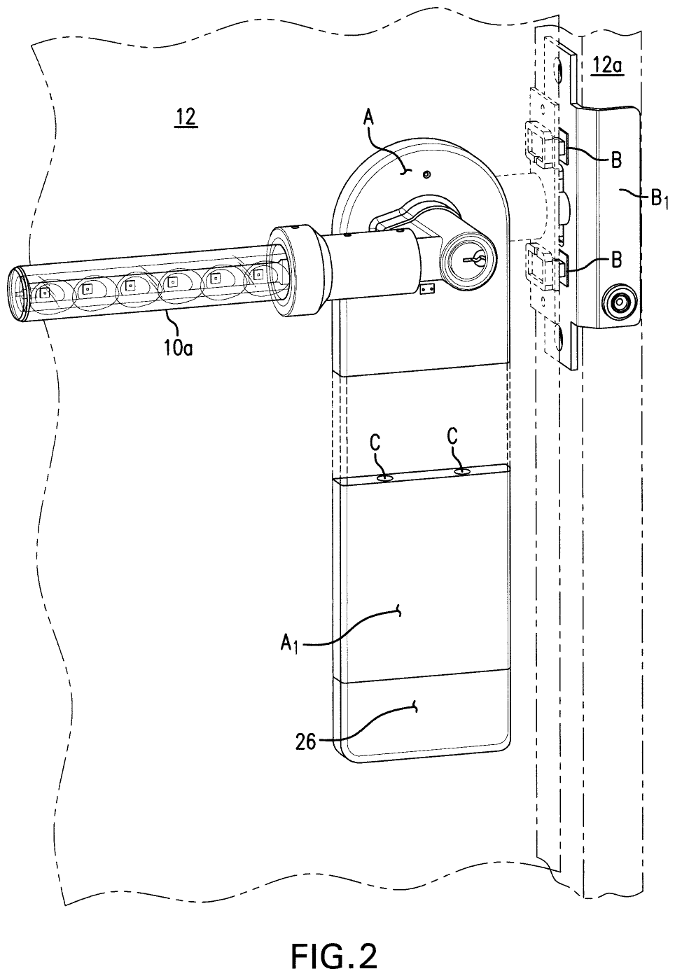

FIG. 2 is a perspective view of the ultraviolet C self-sterilizing germicidal light airborne pathogen killing system A, with assembled module A1 for ultraviolet C self-sterilization of door access devices such as door handles, door levers, door push plates and the like for hand sanitation with system assembled to door 12. Handle 10a may be as illustrated and/or in the shape of a lever and module A1 displaced from UVC door lever system A.

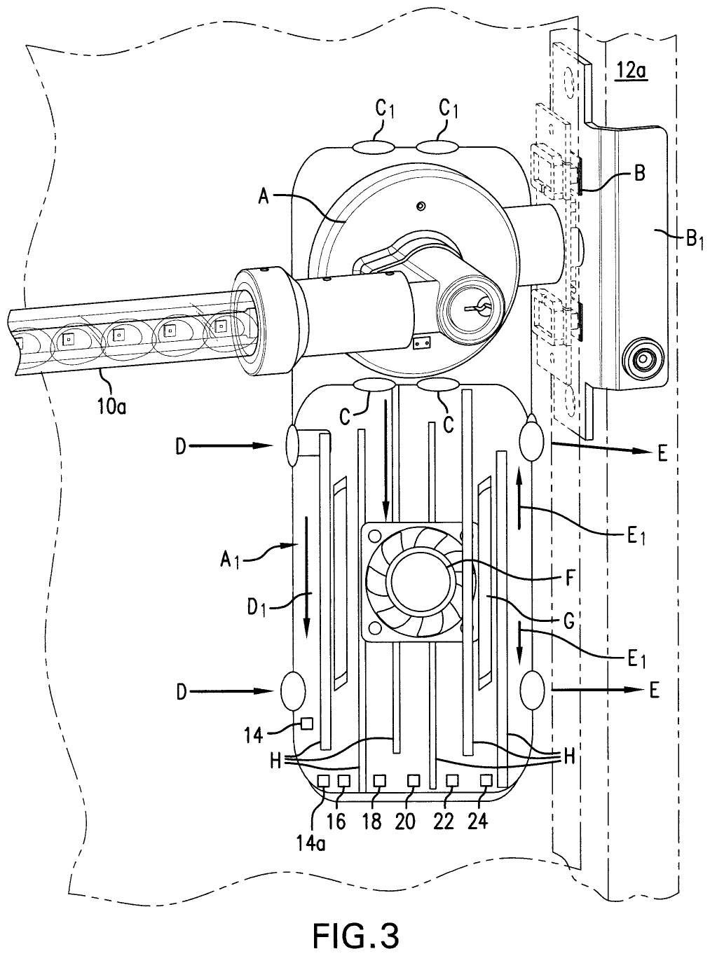

FIG. 3 is an elevation view of the ultraviolet C self-sterilizing germicidal light airborne pathogen killing system, with cut-away view assembled module A1 for ultraviolet C self-sterilization of door access devices such as door handles, door levers, door push plates and alike for hand sanitation and is assembled to door 12. As indicated, C1 module snap-on joiner electrical connecting means ate located on the top of UVC system. Air entry ports D facilities the injection of untreated air D1 circulated via a fan F. Untreated air D1 is directed by baffles G past a source of ultraviolet C self-sterilizing germicidal light H with the resultant treatment of the air and the UVC treated air E1 is evacuated through air exit ports E and onto a person's hands killing the airborne pathogens.

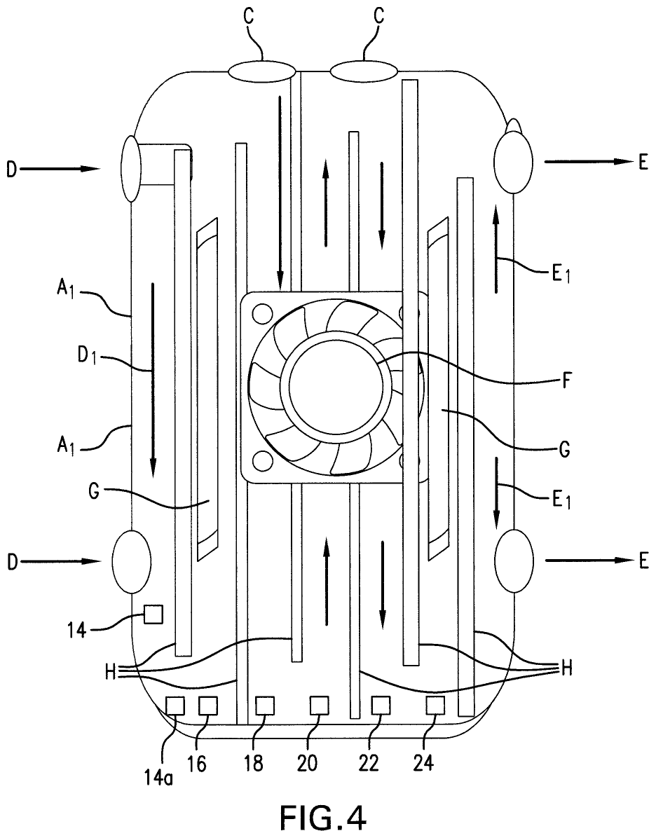

FIG. 4 is an elevation view of the ultraviolet C self-sterilizing germicidal light airborne pathogen killing system, with an enlarged view of module A1 further illustrating its components. Module A1 may be activated by motion sensor 14, optical sensor 14a and/or heat sensor 16. Battery 18 is provided as an alternate electrical energy source for fan F and source of ultraviolet C self-sterilizing germicidal light H. Further provided is camera 20, speaker 22 and in addition a remote program system 24.

Module A1 is functional as a stand-alone device and electrically powered by batteries (DC) 18 and/or source of external AC. The device may be located at the discretion of the installer of said device.

While only several embodiments of the present invention have been shown and described, it will be obvious to those skilled in the art that many modifications may be made to the present invention without departing from the spirit and scope thereof.

1. An ultraviolet C self-sterilizing germicidal light airborne pathogen killing system for ultraviolet C self-sterilization of door access devices such as door handles, door levers, door push plates for hand sanitation, comprising:
a door access device associated with a door jamb;
a module affixed to said door access device;
a source of electricity associated with the door access device;
means for electrically powering said module;
means for the module for sanitizing air for hand sanitization; and,
means for activating said module for hand sterilization;
said ultraviolet C self-sterilizing germicidal light airborne pathogen killing system being modular and field-sizable for filing dimensions of at least one of said door access device and a room to which a door is associated.

2. The ultraviolet C self-sterilizing germicidal light airborne pathogen killing system for ultraviolet C self-sterilization of door access devices according to Claim 1, wherein said door access device comprises:
a handle for operating said door access device; and,
a latching system accessed by said door handle, said latching system being in communication with the door jamb.

3. The ultraviolet C self-sterilizing germicidal light airborne pathogen killing system for ultraviolet C self-sterilization of door access devices according to Claim 1, wherein said module affixed to the door comprises:
a fan to ingest untreated air and expel UVC treated air;
a source of ultraviolet C self-sterilizing germicidal light; and
a baffle to direct ingested air past the ultraviolet C self-sterilizing germicidal light for hand sterilization.

4. The ultraviolet C self-sterilizing germicidal light airborne pathogen killing system for ultraviolet C self-sterilization of door access devices according to Claim 1, wherein the source of electricity associated with the door access device comprises:
electrified contacts in door jamb;
electrical contacts in door so that electric is provided to the door; and
electrical contacts on the door.

5. The ultraviolet C self-sterilizing germicidal light airborne pathogen killing system for ultraviolet C self-sterilization of door access devices according to Claim 1, wherein said for means for electrically powering said module comprises:
electrical contacts in the module;
electrical contacts in door so that electric is provided to the door, and,
communication between the electrical contacts in the module and electrical contacts on the door.

6. The ultraviolet C self-sterilizing germicidal light airborne pathogen killing system for ultraviolet C self-sterilization of door access devices according to Claim 1, wherein said means for said module for sanitizing air for hand sanitation comprises:
an entrance for untreated air;
a source of UVC self-sterilizing germicidal light;
a fan to ingest untreated air;
baffles to direct the untreated air past the source of UVC self-sterilizing germicidal light so that the air may be treated with the UVC self-sterilizing germicidal light; and
an exit for the UVC self-sterilizing germicidal light.

7. The ultraviolet C self-sterilizing germicidal light airborne pathogen killing system for ultraviolet C self-sterilization of door access devices according to Claim 6, wherein the source of UVC self-sterilizing germicidal light is at least one of UVC LEDs, cold cathode tubes and electromagnetic sterilization.

8. The ultraviolet C self-sterilizing germicidal light airborne pathogen killing system for ultraviolet C self-sterilization of door access devices according to Claim 1, wherein said means for activating said module for sanitizing air for hand sanitation is at least one of a heat sensor, an optical sensor, a motion sensor, a passive infrared sensor, a microwave sensor, an ultrasonic sensor, a tomographic motion detector sensor, a video camera sensor and a gesture detector sensor.

9. An ultraviolet C self-sterilizing germicidal light airborne pathogen killing module for ultraviolet C self-sterilization of surrounding ambient air, comprising:
a module to sanitize ambient air enabled with ultraviolet C self-sterilizing LED UVC light emitting technology;
a fan within said module;
modular compatibility for snapping into a door opening system;
means for facilitating a path of ingested air around the ultraviolet C self-sterilizing LED UVC light emitting technology;
means for electrically powering said module; and,
means for activating said module for air sanitation.

10. The ultraviolet C self-sterilizing germicidal light airborne pathogen killing module for ultraviolet C self-sterilization of surrounding ambient air according to claim 9, wherein said module to sanitize ambient air enabled with ultraviolet C self-sterilizing LED UVC light emitting technology comprises a source of UVC self-sterilizing germicidal light including at least one of UVC LEDs, cold cathode tubes and electro-magnetic sterilization.

11. The ultraviolet C self-sterilizing germicidal light airborne pathogen killing module for ultraviolet C self-sterilization of surrounding ambient air according to claim 9, wherein said fan within said module injects unsensitized ambient air and expels sanitized air.

12. The ultraviolet C self-sterilizing germicidal light airborne pathogen killing module for ultraviolet C self-sterilization of surrounding ambient air according to claim 9, wherein said means for facilitating the path of ingested air around the ultraviolet C self-sterilizing LED UVC light emitting technology are baffles for facilitating air circulation.
